(19) United States
(12) Patent Application Publication
(10) Pub. No.: US 2007/0084462 A1
(43) Pub. Date: Apr. 19, 2007

(54) Counting device

(76) Inventors: Jeffrey K. Allen, Cheshire (GB); Naseem Bari, Manchester (GB)

Correspondence Address: STITES & HARBISON PLLC, 1199 NORTH FAIRFAX STREET, SUITE 900, ALEXANDRIA, VA, 22314, US

(21) Appl. No.: 10/571,681

(22) PCT Filed: Sep. 10, 2004
(86) PCT No.: PCT/GB04/03894

§ 371 Date: Nov. 29, 2006

(30) Foreign Application Priority Data
Sep. 11, 2003 (GB) 0321296.6

(51) Int. Cl. A61M 15/00; A61M 11/00

(52) U.S. Cl. 128/200.23; 128/203.15

(57) ABSTRACT

The present invention provides a counting device (100) capable of recording the number of doses dispensed from dispensers such as inhalers. The counting device is mounted on a portion of an inhaler canister (200) protruding from a housing (300), at an end opposite a delivery portion of the canister. The inhaler canister is mounted in the housing to form the dispenser. The counting device includes a switch (3) actuatable by interaction with the housing during relative movement between the canister and the housing. A display means (7) is provided for displaying information related to the number of doses delivered by the dispenser.

The present invention relates to a counting device capable of recording the number of doses dispensed from dispensers such as inhalers, e.g. from Pressurised Metered Dose Inhalers (PMDIs).

Inhalers are commonly used to administer medication such as bronchodilators, corticosteroids and anti-allergenics to the pulmonary tracts of humans and other animals, e.g. to treat nasal and bronchial complaints such as asthma.

A typical inhaler includes a housing or body and a canister containing the drug. The canister may be an integral part of the inhaler, but more commonly is removable, so that the actuator may be cleaned. In most applications, the canister contains the drug in powder or liquid form, and the dispensing action forces out a regulated dose of the drug in aerosol form.

The aerosol may be created using a propellant e.g. CFC or HFA forced through the drug by the dispensing motion, or more preferably in PMDIs by creation of a significant overpressure inside the canister.

In most PMDIs, the dispensing action consists of moving the canister relative to the housing, the action of part of the housing on the nozzle of the canister causing a predetermined dose to be released when the nozzle has been depressed a set distance.

Typical canisters for use in PMDIs contain between 60 and 200 doses, and may be administered either on a regular (e.g. daily) basis or only occasionally to relieve particular symptoms. Even in the former case, the user is unlikely to keep a record of the doses administered, so will not be aware when the canister no longer contains any drug. The user will not necessarily be immediately aware that the drug is no longer being dispensed as the pressure of the canister, or the dispensing motion, will appear unaltered. In an extreme situation, this could result in a user who needs a dose in an emergency finding that their inhaler had in fact been exhausted in earlier use.

Therefore, there is a need for some form of counting device which can count the number of doses dispensed from a canister and so provide an indication of when the canister is empty. In order to be of use, such a counting device must accurately record each actuation of the canister.

Previous attempts to provide such a counting device have had a number of drawbacks. In particular, some solutions have been proposed with a counting device which is mounted on the housing of the inhaler and records each actuation of the canister using, for example, a pressure pad or a lever based switch. These devices suffer from the problem that they either have to be reset manually or, if a canister is removed from the inhaler, the counter automatically resets, so if the same canister is replaced (e.g. after cleaning), the reading will be incorrect.

To overcome this problem, other solutions have proposed a counter mounted on the “top” end of the canister (the opposite end from the dispensing nozzle and normally the part which the user presses to use the inhaler), and includes a pressure-activated switch which increments the counter.

These latter devices do not provide particularly accurate indications of the number of doses dispensed from or remaining in the canister. This is because the pressure required to close the pressure switch is rarely exactly the same as that required to operate the inhaler to dispense the drug. Indeed, the pressure required to move the nozzle the set distance for dispensing the drug may vary considerably, both from canister to canister and indeed during the life of each canister as the pressure inside the canister changes. Therefore, there is significant potential for either under- or over-counting the doses actually delivered, which can result in users believing that the inhaler is still dispensing the drug when it has in fact run out, or in canisters which still contain several doses of the drug being disposed of because they are indicated as being exhausted. Moreover, canisters with top-mounted counters may be actuated without pressure being put on the counter (e.g. the canister may be gripped around its edges), therefore a dose could be obtained from such dispensers without activating the counter.

To attempt to overcome some of the above problems, EP 0986412 (Glaxo Group Limited) teaches a counter attachment for the nozzle end, of a canister. The attachment can be removed with the canister so that the dose information is not lost. Furthermore, because the attachment is located near the nozzle, the counter is actuated by the relative movement of the canister with respect to the housing. However, the attachment proposed in that document requires wholesale redesign of the housing in which the canister is mounted. Moreover, fitting the attachment around the nozzle is a relatively difficult process. Indeed, it is inconvenient to have the counter at the ‘business’ end of the canister, because all of the critical components have to be squeezed in to a small space.

At its most general, the present invention proposes a counting device for mounting on a canister for use in a dispenser (e.g. an inhaler), wherein the counting device is not mounted at the nozzle end of the canister yet the dispenser cannot be actuated without causing actuation of the counting device.

According to a first aspect of the present invention, there is provided a dispenser for dispensing a dose of a substance, the dispenser having: a canister for containing the substance, the canister having a delivery portion at one end which includes delivery means through which a dose is deliverable, the delivery portion being mounted in a housing such that relative movement between the canister and the housing actuates the dispenser, and a protruding portion at an end opposite the delivery portion which protrudes from the housing; a display means for displaying information related to the number of doses delivered by the dispenser; and an electrical counting device attached to the protruding portion, the counting device including a switch, wherein the switch is actuatable by interaction with the housing during relative movement between the canister and the housing.

Preferably either the counting device includes the display means or the display means is included in a display module attached to the housing, preferably to the outside of the housing.

The counting device is therefore not wholly enclosed in the housing. This means that the device may be used with existing housings, thereby removing the need for substantial redesign. Moreover, the compact nature of many e.g. inhaler dispensers may be maintained because the counting device need not be located in the housing of the dispenser. The present invention may be particularly useful in inhaler dispensers. The shape of an inhaler canister is also well known, and the counting device may be adapted to fit such a shape.

Furthermore, the invention proposes a counting device which is activated by an interaction with the housing during relative movement of the canister with respect to the housing. This reduces the potential for false counting, because it may be impossible to get a dose from the dispenser without causing the switch on the counting device to be actuated. This is different from the prior art, where e.g. a certain pressure on the counter switch caused it to be actuated and attempts were made to make that pressure the same as the pressure required to activate the dispenser. In that case, the counter was actuated by an interaction with the user. This meant that it was still possible for the user to activate the dispenser without having to press the counter switch. In the present case, the user need only activate the dispenser and the counting device will automatically be actuated. Actuating the dispenser is the movement required between the housing and the canister for a dose to be delivered from the delivery means of the canister. This may be caused by a protrusion in the housing engaging the delivery means of the canister. Preferably, the canister is an inhaler canister and the dispenser is an inhaler, so the delivery means is preferably a nozzle.

Having the display means included in the display module attachable to the housing is preferable when, for example, a user's view of the display means would be obstructed if the display means were instead included in the counting device attached to the canister. For example, the dispenser may include a cover or additional housing, for covering up the protruding part of the canister (and therefore the counting device) to prevent unintentional actuation of the dispenser when not in use. However, the user may still desire to check information displayed by the display whilst the cover is in place. So, having the display means included in a display module attached to, e.g. the outside of the housing, may allow such a check to be made.

The electrical counting device may be electronic. Preferably, the electrical counting device includes an integrated circuit. The integrated circuit may include an electronic counter. Preferably the counting device includes a flexible substrate, or a printed circuit board (PCB) on which electronic components of the counting device can be placed. Preferably the PCB includes contacts for connecting to the switch. Preferably the electrical counting device includes a memory. Preferably the memory is a memory media integrated chip. Preferably the memory may store any kind of data information. Preferably the memory stores items of information useful to the user of the dispenser, for example, preferably the memory stores: an Identification code (ID) specific to the canister to which it is attached (i.e. to identify the canister); a record of the initial amount of drug stored in the canister before use of the canister for the first time; a record of the number of doses of drug dispensed from the canister; and/or a record of the amount of drug remaining in the canister.

The display means may be connected to the electronic counter and/or memory. Preferably the display means may display one or more of the items of information stored in the memory.

Preferably, the counting device is attached only to the protruding portion. Preferably, the counting device remains outside the housing during actuation. This makes it even easier to apply the counting device to known dispensers.

Preferably, the switch is actuatable by interaction with the top of the housing. In other words, when the dispenser is assembled and it at rest, the actuating portion of the canister will protrude above the top edge of the housing. To activate the dispenser, the user may push the canister further into the housing. Thus, part of the protruding portion moves relative to the top edge of the housing. The present invention utilises this movement to actuate the switch.

The switch may include a downwardly facing button displaceable by a physical interaction with the top of the housing during relative movement between the canister and the housing. Thus, as the canister is depressed into the housing, the top edge will move upwards relative to the canister and engage the downwardly facing button. This arrangement means that no balancing of pressures required to activate the dispenser and the counting device is required. The distance of relative movement between the housing and the canister required to dispense a dose is known and may not vary. Therefore the button is positioned so that movement that will result in dispensing a dose will automatically register a count.

The switch may include a reed switch actuatable by a magnetic interaction with the top of the housing during relative movement between the canister and the housing. In this case, the switch need not even touch the housing. This is another advantage if the counting device is required to be applied to a known dispenser. The housing preferably has a magnetic strip mounted on it. This is preferably near or on the top edge of the housing in the vicinity of the switch. Again the distance that the reed switch needs to be moved to be actuated by the magnetic strip needs to be balanced with the amount of relative movement required between the housing and the canister to dispense a dose.

The housing may include a finger portion extending from its top edge for interacting with the switch. Thus the finger portion may be the only part of the housing that even comes close to the counting device.

The switch may include a button displaceable by a physical interaction with the finger portion during relative movement between the canister and the housing. Thus, the finger portion may press against the downwardly facing button mentioned above, or the button of the switch may be displaceable in e.g. a radial direction. In this case, the finger portion may overlap with the button so as to push it towards the body of the canister. The finger portion may include a dimple for engaging the button and forcing it e.g. radially inwards.

Alternatively, the switch may include a reed switch actuatable by a magnetic interaction with the finger portion during relative movement between the canister and the housing. In this case, the top of the finger portion may have a magnetic strip mounted on it, or a magnetic part may be located inside the finger portion such that the counting device is activated when the finger portion overlaps with the reed switch.

The counting device may be mounted on a side wall of the protruding portion of the canister. The device may be in the form of a casing mounted on a flexible substrate which may be stuck to the wall of the canister. The counting device may be self-contained so that it can be applied as one piece to the canister.

Preferably, the counting device includes a flexible casing for holding the components of the device, the flexible casing being mounted on the flexible substrate for encapsulating the components of the device. This provides an extra advantage from known devices where the casing was rigid; having a flexible casing and a flexible substrate allows the device to be mounted on canisters having many different shapes, because the flexible nature of the counting device means it can bend round canisters having different or even irregular radii.

The counting device may include a battery, preferably a card battery. The card battery may be formed as part of e.g. the flexible substrate of the counting device. This makes the complete counting device more versatile. However, when the display means is included in a display module attachable to the housing, preferably the display module includes the battery.

However, the device and/or display module may also be used with the known type of button battery. The counting device may include a main body for mounting on the end of the protruding portion of the canister (e.g. the circular top face of known inhaler canisters) and a leg portion depending from the main body e.g. for mounting on the side wall of the protruding portion of the canister. The main body may be shaped to contain a button-shaped battery. Mounting the main part of the device on the top end of the canister is yet another advantage if the device is to be applied e.g. to known inhalers. The overall shape of the canister may be less affected if the main part of the device is kept away from the side walls of the canister.

Preferably, the switch is on the leg portion so that it can be closer to the part of the housing with which it is to interact.

The display means may also be on the leg portion.

Preferably, when the display means is included in the display module, the display is connected to the counting device via a communications link. Preferably the communications link can transmit data and/or power. Preferably the display module includes a processor. Preferably, the communications link connects the display and/or processor to the electronic counter and/or memory of the counting device. The communications link may include a removable communications cable, e.g. a wire, flexi, or a rigid flexi. Additionally, or alternatively, the communications link may include clips located on the canister, that electrically contact with metal tracks on the inside of the housing. Preferably the display module has an external interface, forming part of the communications link. Preferably the external interface is a four-way connector. Preferably, the processor detects the presence of a canister, when the display is connected to the counting device via the communications link. Preferably, upon detection of the canister, the display means displays items of information stored in the memory of the counting device. Preferably, if the canister is removed from the housing, the connection via the communications link, between the display module and the counting device, is broken, e.g. the electrical contact between the clips and the metal tracks is cut off. Preferably, if the connection is broken, the display means shuts down, i.e. it displays no information.

Preferably, when the display means is included in the display module, the display module includes the battery. Preferably, the battery provides power to component parts of the display module, e.g. the processor and/or display, and preferably also power, preferably via the communication link, to the component parts of the counting device. Preferably, when the processor does not detect the presence of a canister, power supply to the communications link is halted. This may provide a safety measure. For example, with this arrangement, the counting device and/or display module may be immersed in water, when not connected via the communications link, without significant power drainage or a power short circuit occurring.

According to a second aspect of the present invention, there is provided a canister (e.g. an inhaler canister) for mounting in a housing to form a dispenser, the canister having: a delivery portion at one end which includes delivery means through which a dose is deliverable, the delivery portion being mountable in the housing such that relative movement between the canister and the housing actuates the dispenser, the dispenser including a display means for displaying information related to the number of doses delivered by the dispenser; and a protruding portion at an end opposite the delivery portion for protruding from the housing; wherein the canister has a counting device attached to the protruding portion, the counting device including a switch; wherein the switch is actuatable by interaction with the housing during relative movement between the canister and the housing.

Preferably either the counting device includes the display means or the display means is included in a display module attached to the housing.

The canister may be reversibly mounted in the housing. The counting device may be such to allow the removal of the canister without the electronic counter resetting. Thus, the canister can be removed from the housing e.g. to allow cleaning without losing the data e.g. displayed by the display means. The dispenser may of course be of the type mentioned in the first aspect above. Likewise, any of the other above-mentioned features of e.g. the counting device may be incorporated into the canister of the second aspect.

According to a third aspect of the present invention, there is provided a counting device for mounting on a canister to form a canister according to the second aspect.

The counting device may be arranged to count upwards or downwards, and may have respectively a fixed upper limit or a fixed starting number. Alternatively, the attachment may allow the upper limit or the starting number to be set for each canister.

Preferably, the display means provides at least a warning when a number of doses equal to the upper limit or the starting number have been dispensed. Alternative warning means may also be used.

Particular examples of the display means are a liquid crystal display (LCD) which may display the number of doses dispensed and/or remaining, and may display other information as well, and/or light emitting diodes (LEDs), which may indicate one or more states of the canister (e.g. full, in use, nearly empty and/or empty) according to the patterns displayed.

According to a fourth aspect of the present invention, there is provided a display module, including a display means, for attaching to a housing to form a dispenser according to the first aspect.

The use of a reed switch in a counting device for an inhaler is an independent idea in its own right. Therefore, in a further aspect of the invention, there is provided a counting device for use with a dispenser to count the doses dispensed from the dispenser, wherein the counter includes a reed switch.

Embodiments of the invention will now be described with reference to the accompanying drawings, in which:

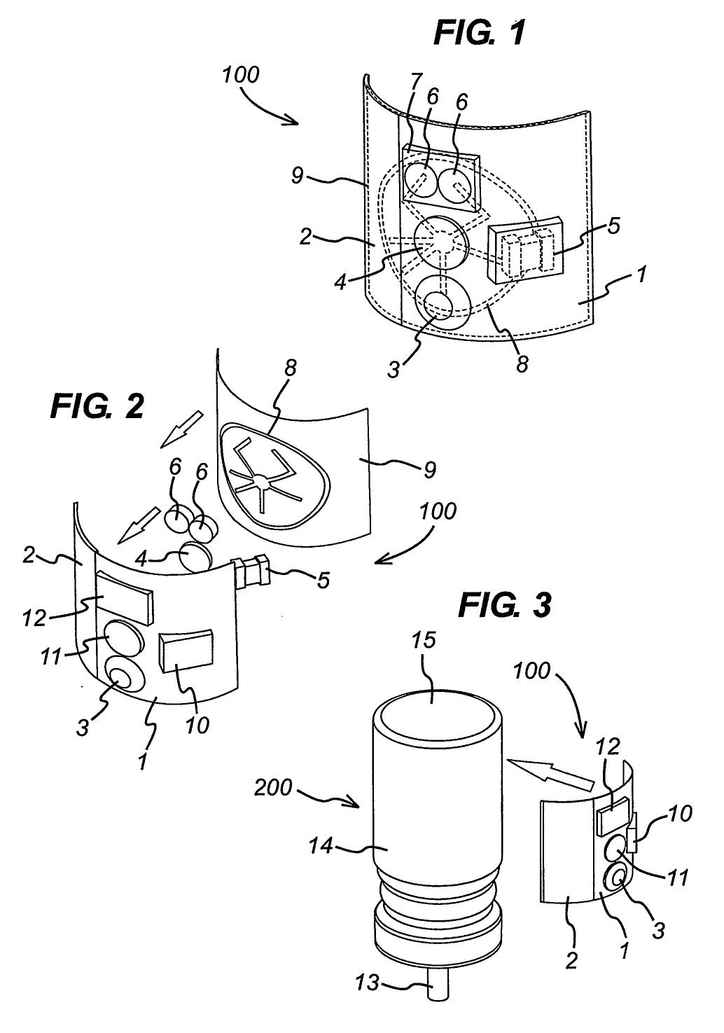

FIG. 1 shows a counting device suitable for use in a first embodiment of the invention;

FIG. 2 shows an exploded view of the counting device of FIG. 1;

FIG. 3 shows the counting device of FIG. 1 being mounted on an inhaler canister;

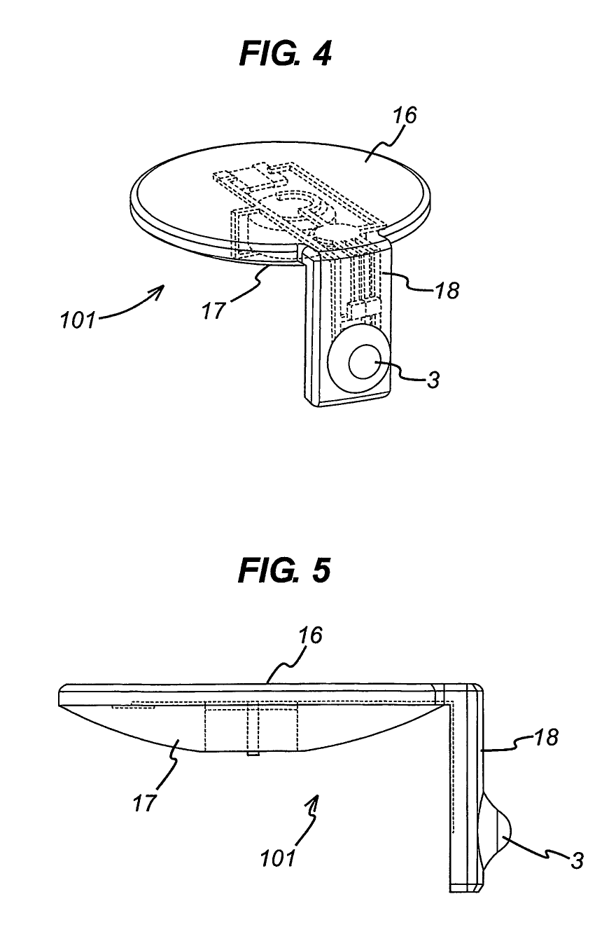

FIG. 4 shows a counting device suitable for use in a second embodiment of the invention;

FIG. 5 shows a side view of the counting device of FIG. 4;

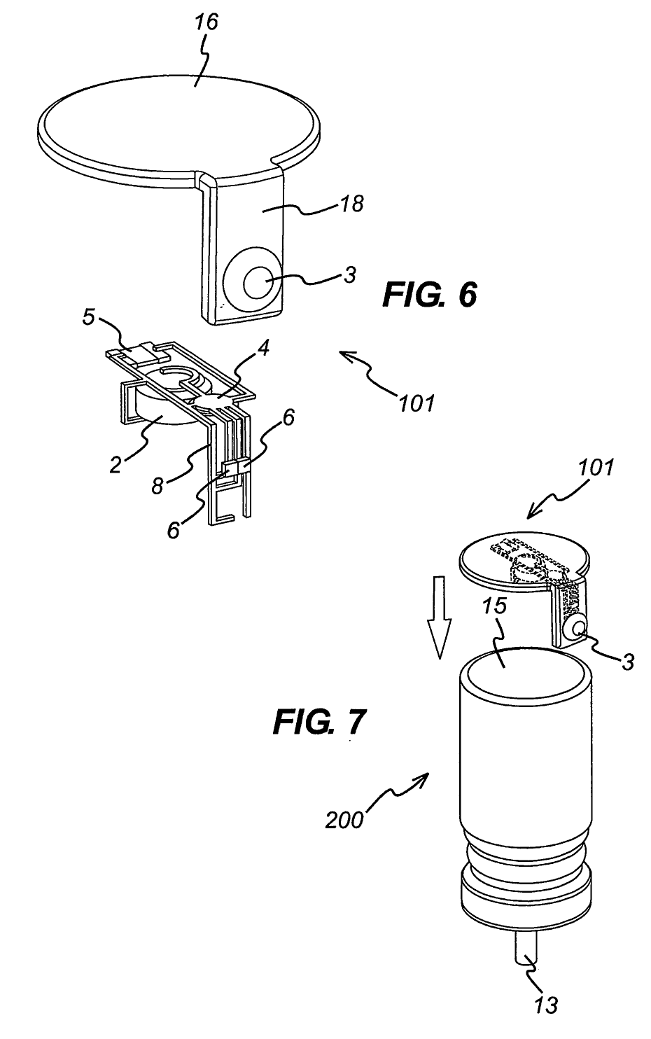

FIG. 6 shows an exploded view of the counting device of FIG. 4;

FIG. 7 shows the counting device of FIG. 4 being mounted on an inhaler canister;

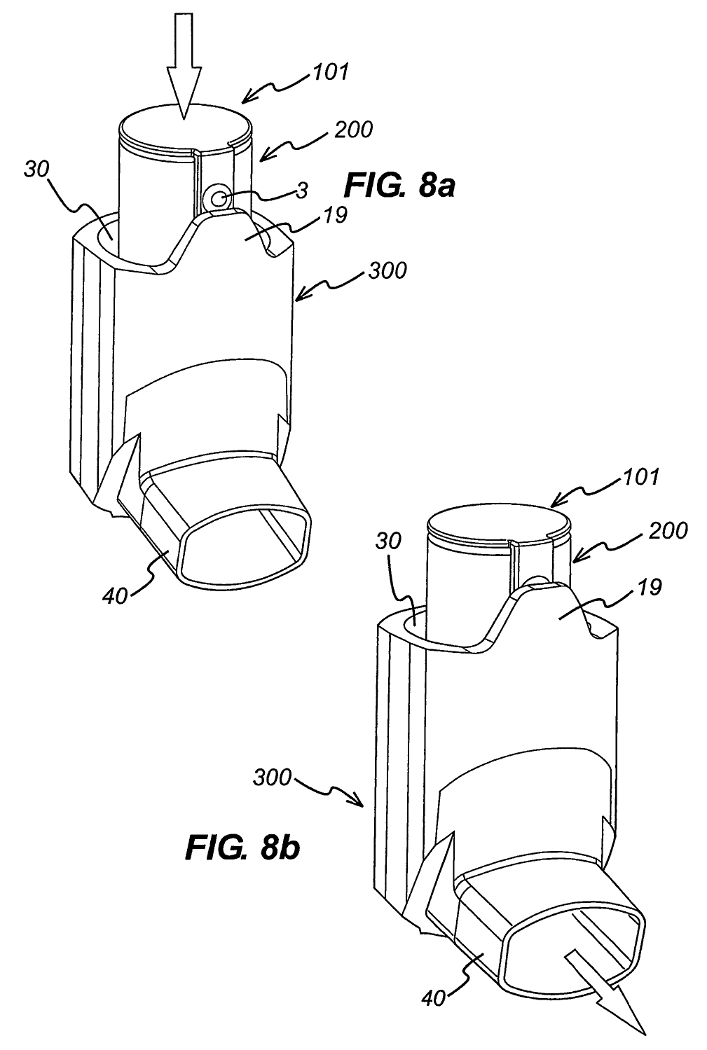

FIGS. 8a and 8b show a dispenser which is the second embodiment of the invention with the counting device of FIG. 4;

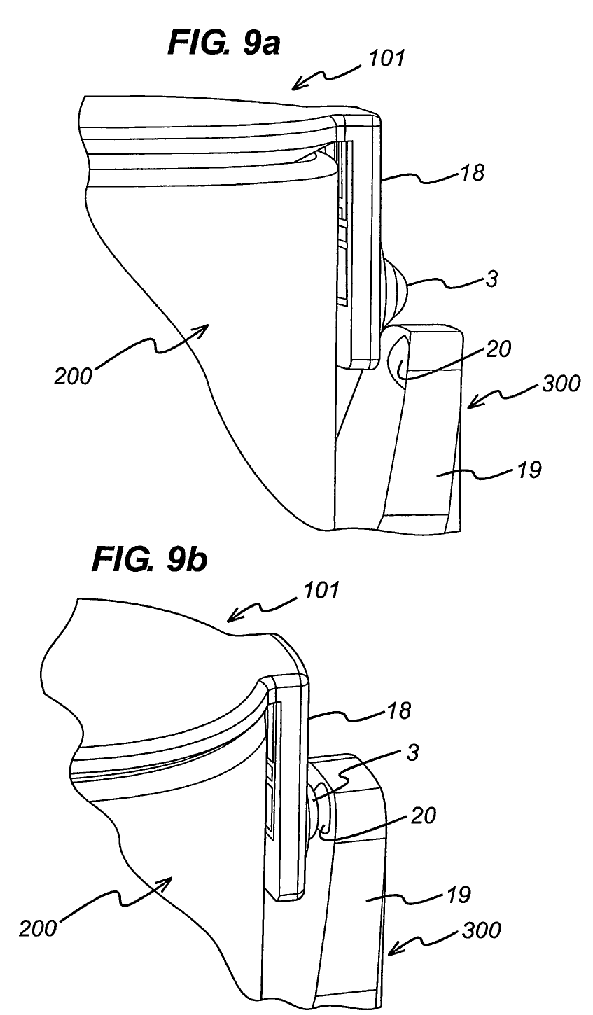

FIGS. 9a and 9b show a close-up side view of the switch mechanism of the dispenser of FIGS. 8a and 8b;

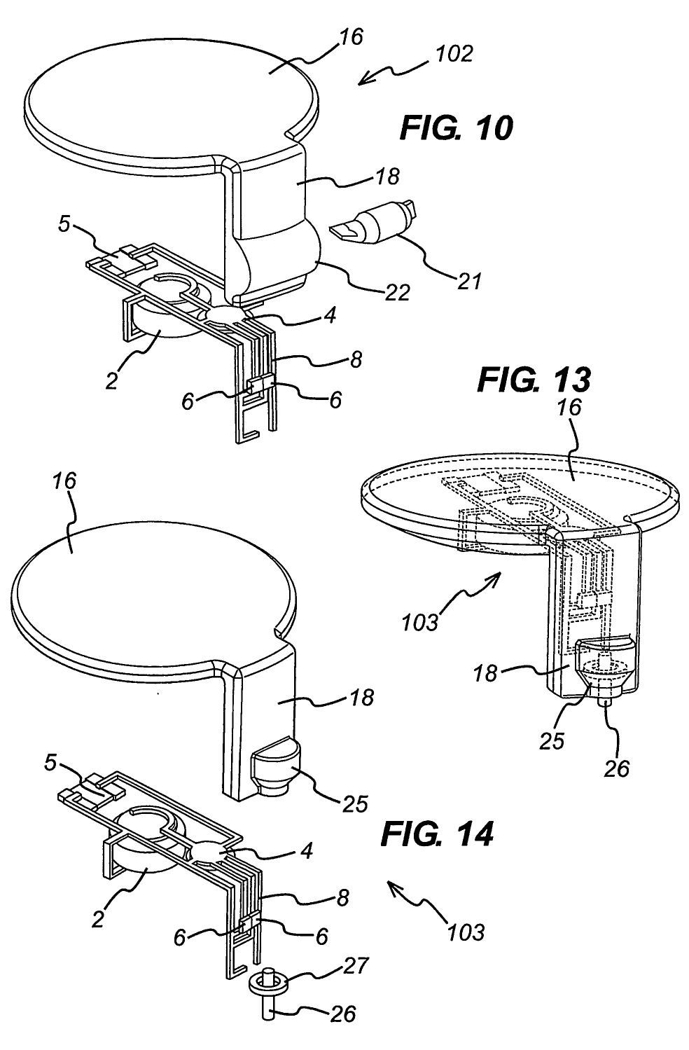

FIG. 10 shows an exploded view of a counting device suitable for use in a third and fourth embodiment of the invention;

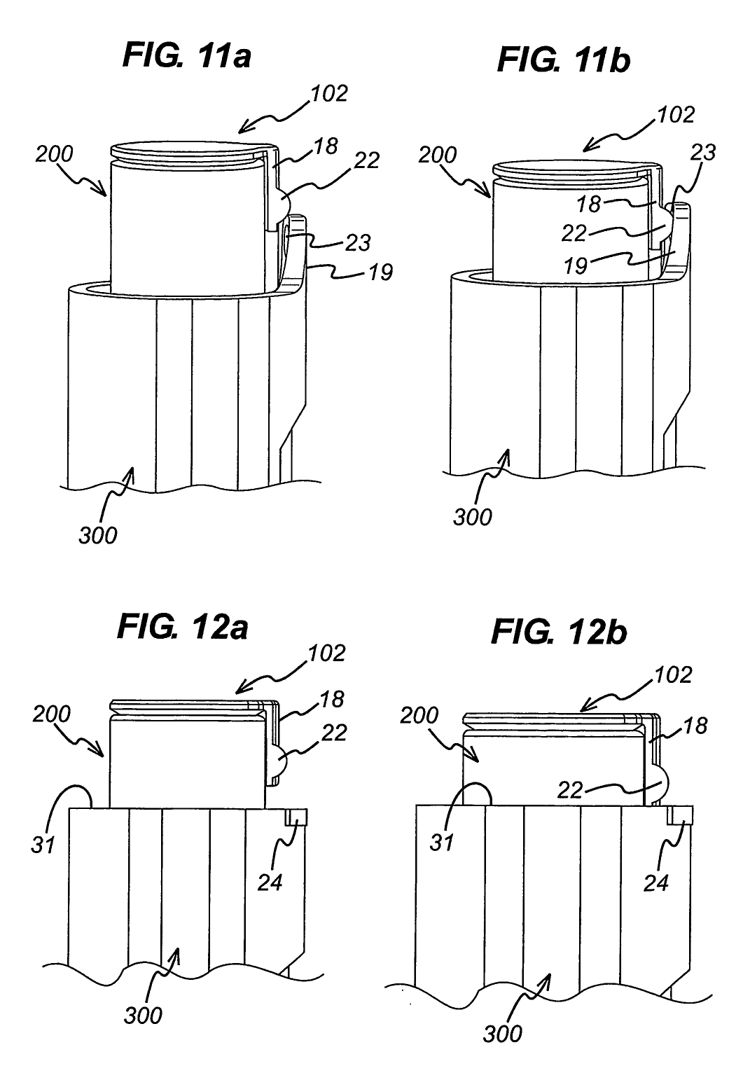

FIG. 11a and 11b show a side view of the switch mechanism of a dispenser which is a third embodiment of the invention;

FIGS. 12a and 12b show a side view of the switch mechanism of a dispenser which is a fourth embodiment of the invention;

FIG. 13 shows a counting device suitable for use in a fifth embodiment of the invention;

FIG. 14 shows an exploded view of the counting device of FIG. 13;

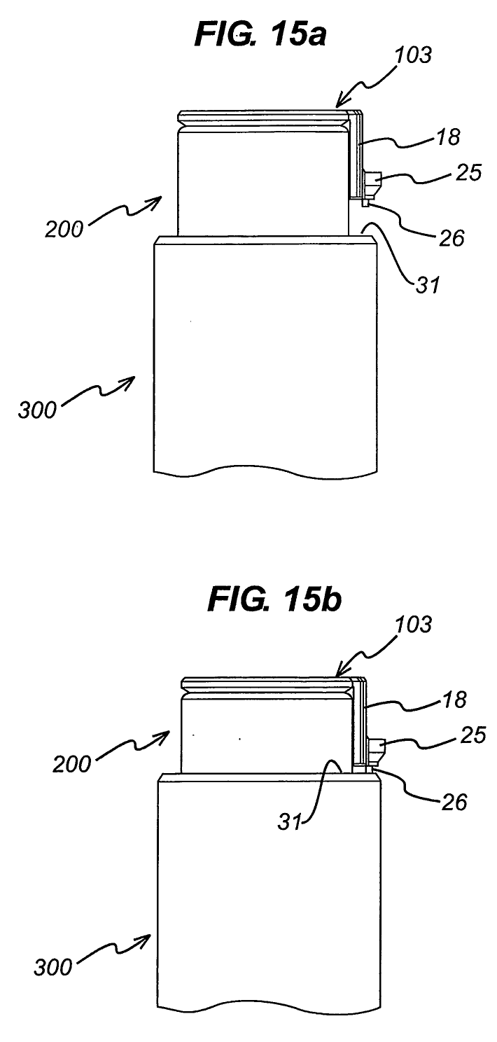

FIGS. 15a and 15b show a side view of the switching mechanism of a dispenser which is a fifth embodiment of the invention.

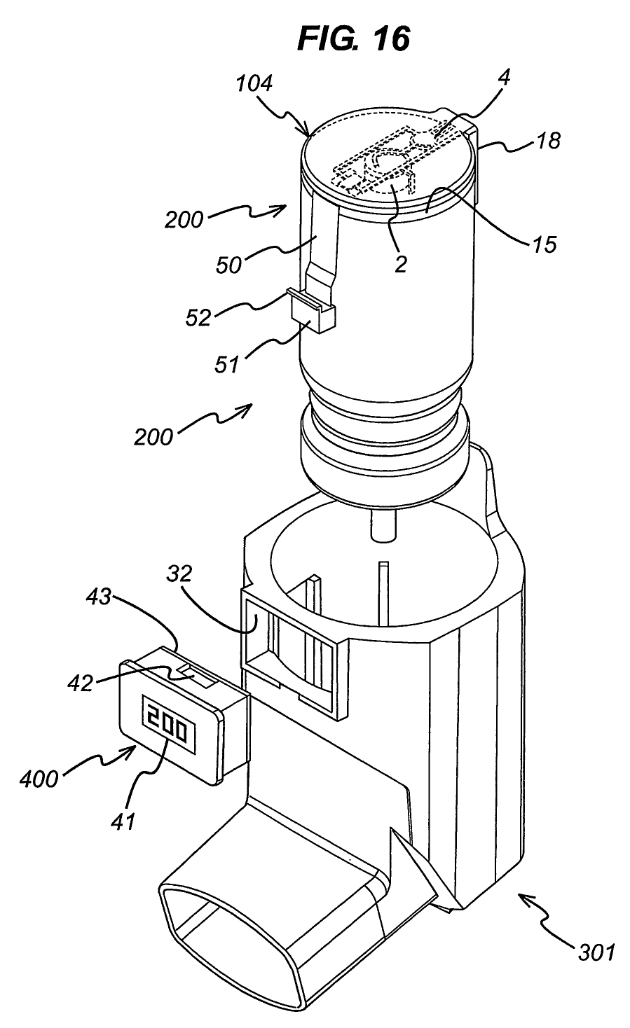

FIG. 16 shows an exploded view of a dispenser including a counting device and display module which is a sixth embodiment of the invention;

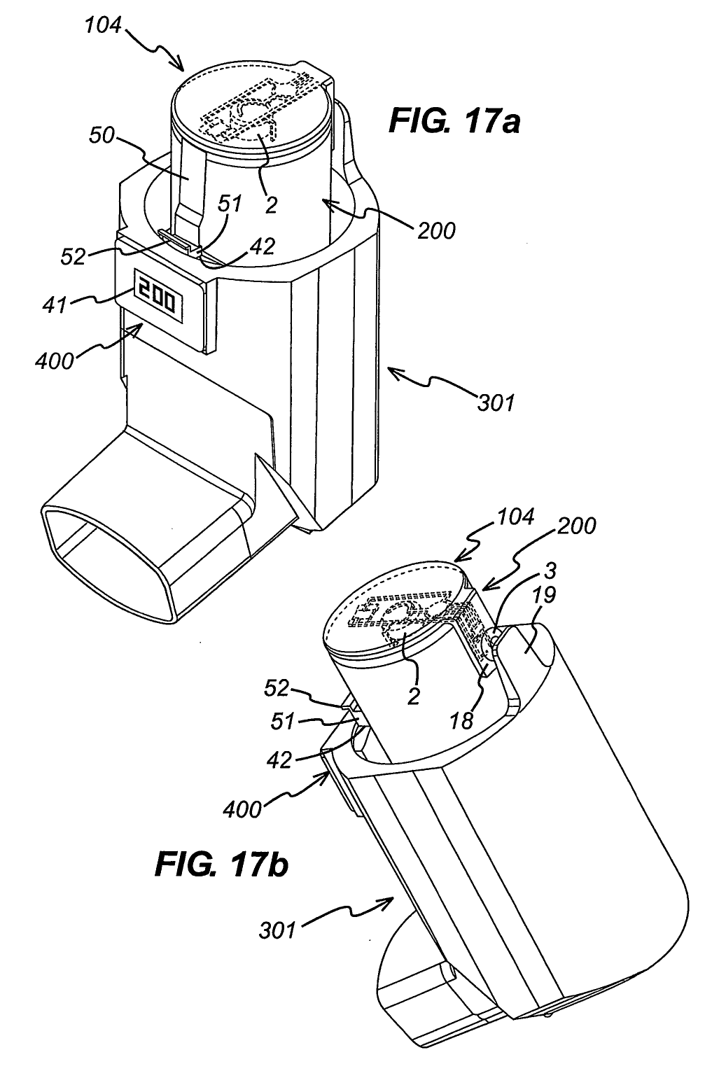

FIGS. 17a and 17b show a front and rear view respectively of the dispenser of FIG. 16; and

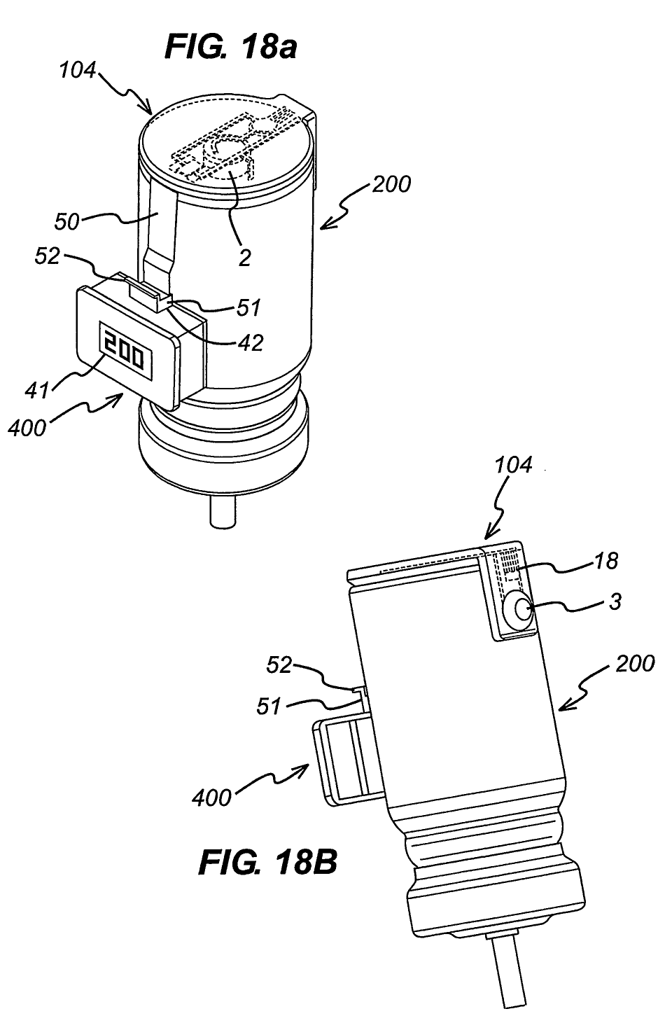

FIGS. 18a and 18b show a front and rear view respectively of the dispenser, without a housing, of FIG. 16.

FIG. 1 shows a counting device 100 that is suitable for attaching to the side of an inhaler canister for mounting in a housing to form a dispenser. The counting device 100 has a front casing unit 1 which holds the units necessary for the functioning of the device. A card battery 2 is connected to the front unit 1 to provide power to the device. The card battery 2 allows the device to have a substantially flat shape, which means it can be mounted on known canisters without altering their overall shape too much. The card battery 2 may clip into the device 100, or it may be integrally formed as part of the rear substrate 9. The front unit 1 holds an application specific integrated circuit (ASIC) 4, which may provide the actual counting mechanism of the device, a button switch 3 for activating the counter, a display panel 7 having e.g. two LEDs 6, and a capacitor 5. Although LEDs 6 are illustrated, an LCD display could also be used. As shown in FIG. 2, these electronic components are located in pockets formed in the front unit 1. The LEDs 6 are located in display pocket 12, the ASIC 4 is located in processor pocket 11, and the capacitor 5 is located in pocket 10.

The electronic components are encapsulated into their respective pockets by fixing (e.g. gluing) the rear substrate 9 to the front unit 1. The rear substrate 9 contains tracking 8 made of a suitable conducting material (e.g. copper) for providing the necessary connections between the electronic components. Either one or both of the front unit 1 and the rear substrate 9 may be made from flexible material (e.g. polyamide) such that the curvature of the counting device 100 may be varied to suit different sizes of canister.

FIG. 3 shows the counting device 100 being attached to the side wall 14 of a typical inhaler canister 200. The counting device is attached at the ‘top’ end of the canister 200, i.e. near to the top surface 15 opposite the nozzle 13. This means that the button 3 and indeed the rest of the counting device need not be located in the housing of an inhaler. Counting device 100 may be operated in a similar way to that shown in FIGS. 8a and 8b, which is explained in more detail below. A dispenser having the canister and counting device shown in FIG. 3 is a first embodiment of the invention and is not illustrated.

FIG. 4 shows a second type of counting device 101, which is suitable for mounting on the top surface 15 of an inhaler canister. Components which are the same as in counter 100 are given the same reference numbers. The device 101 has a lid 16 for covering the electronic components, which are contained in top unit 17 shown in FIG. 5, which may have pockets for holding the individual components. The lid 16 may be sealable to the top unit 17, whereby each counting device is a disposable single item, i.e. it is not intended to replace the battery. The device 101 has a leg portion 18 which depends from the edge of the top unit 17. The leg portion 18 may be positioned so that it lies against the side wall of the canister when the top unit 17 is fixed (e.g. glued) to the top surface 15. The leg portion 18 may contain the button switch 3 and/or the display indicators, which are LEDs 6 in this case, but could also be a LCD display.

FIG. 6 shows in more detail the electronic components that are inserted into the top unit 17. In this case, a coin cell or button battery 2 of the well known type (e.g. of the type made by Varta) may be used. Moreover, the tracking 8 may be separate from both the top unit 17 and lid 16.

As shown in FIG. 7, the counting device 101 may be attached (e.g. using adhesive) to the top surface 15 of an inhaler canister 200. The shape of the top section of the counting device preferably matches the shape of the canister 200, so that again the overall shape of the canister is not substantially affected by attaching the counting device 101. As many known canisters have an indentation in their top surface 15, the pockets containing the electronic components in top unit 17 may fit snugly against the canister such that the lid 16 can be flat.

FIGS. 8a, 8b, 9a and 9b illustrate the operation of a dispenser according to a second embodiment of the invention which includes counting device 101. However, the principles of operations will be appreciated as applying to other types of counting device, e.g. the device 100 illustrated in FIG. 1. FIGS. 8a and 8b show a typical inhaler housing 300 having a mouthpiece 40 at one end, and a canister 200 mounted in an aperture 30 at the other end. The canister 200 is mounted so that a portion of it protrudes from the housing 300. To activate the dispenser (e.g. inhaler), the canister 200 is depressed so that it moves downwards into the housing 300. The action of e.g. a protruding member (not shown) in the housing on the nozzle causes a dose to be dispensed from the nozzle out through the mouthpiece 40. In FIGS. 8a and 8b, the housing 300 has a finger portion 19 extending upwards (i.e. away from the mouthpiece end) from its top edge 31. As shown in FIGS. 9a and 9b, the inside of the finger portion 19 has a dimple 20 made of rigid material or formed as part of the housing itself which engages the button switch 3 as the canister 200 moves down relative to the housing 300 to dispense a dose. The finger portion 19 and/or the shape of the canister 200 or aperture 30 may be such that the canister cannot be depressed to deliver a dose without the button switch 3 being actuated by the dimple 20. Therefore the dispenser will automatically register a count whenever a dose is delivered through the mouthpiece 40.

FIG. 10 shows a further example of a counting device 102 for use in the present invention. Again, like components have been given the same reference numbers. This counting device is similar to the one shown in FIG. 6 except that instead of a button switch 3 it has a reed switch 21 mounted behind a cover 22 in the leg portion 18. Using the reed switch 21 presents potentially new ways of actuating the counter. FIGS. 11a and 11b show the top of a dispenser which is a third embodiment of the invention. Counter 102 is attached to the top surface 15 of canister 200 in a similar way to counter 101 in FIG. 7. The housing 300 again has a finger portion 19 extending upwards from its top surface so that the cover 22 encasing the reed switch 21 overlaps with the end of the finger portion 19 as the canister 200 is depressed to deliver a dose. In this embodiment, instead of the finger portion 19 including a dimple 20, it includes a magnet 23 which causes the reed switch 21 to activate when it is within a certain distance. This distance can be set so that the dispenser cannot be activated without the counting device registering a count.

FIGS. 12a and 12b show a fourth embodiment of a dispenser according to the invention. In this case, no part of the counting device 102 enters the housing 300. The advantages of this are clear: it allows the counting device to be applied to known shapes of canister and housing without the need for wholesale redesign of the same. Instead, a magnetic strip 24 need only be mounted on or near the top edge 31 of the housing 300 in the vicinity of the reed switch cover 22 when the canister is depressed. The magnetic strip 24 may be made out of any well known magnetic material, and may be flexible so that it can be adhered (e.g. using glue) to the housing. As in FIGS. 11a and 11b, the distance between the magnetic strip 24 and the reed switch 21 is set so that the dispenser cannot be activated without the counting device recording a count.

FIG. 13 shows a further example of a counting device 103 for use in the present invention. As can be seen from FIG. 14, it is similar in construction to the counting devices shown in FIGS. 6 and 10. The difference in this case is that the counting device 103 has a switch 26 for directly engaging with the top surface of the dispenser housing. Again, the switch 26 is contained in a cover 25 on the leg portion 18 of the counting device 103, although other positions may be possible. The switch 26 faces e.g. downwards so that it moves e.g. substantially parallel to the direction in which the canister is pushed to deliver a dose. The switch 26 is held in a ring-like guide 27 for support and to make sure it moves in the correct direction.

FIGS. 15a and 15b show the top of a dispenser according to a fifth embodiment of the invention, having counting device 103 mounted on canister 200 in a similar way to that shown in FIG. 7. When the canister is depressed in the housing so that it moves downwardly relative to it, the switch 26 is brought into engagement with and actuated by the top edge 31 of the housing 300. As the distance the canister needs to be depressed to deliver a dose is known, the position of the switch 26 (determined e.g. by the length of the leg portion 18) can be set so that the dispenser cannot be activated without the counting device registering a count. The fifth embodiment shares many advantages with the fourth, in that it can clearly be applied to e.g. inhaler housings that are already constructed, because no part of the counting device needs to enter the housing.

FIGS. 16, 17a, 17b, 18a and 18b show a further example of a counting device 104, and a display means, suitable for use in the present invention. As can be seen from these Figures, the counting device 104 may be similar in construction to the counting device 101 of FIGS. 4 to 9.

With reference to FIG. 16 in particular, a difference is that the counting device 104 may not include the display means. Instead the display means, such as an LCD display 41, may be provided in a display module 400, that can be mounted in e.g. an opening 32 provided in a housing 301 similar to the housing 300 shown in e.g. FIG. 8a. The display module may be e.g. glued or snap fit into place in the opening 32. The display module may have a cushion layer 43 at its rear to provide a soft surface for abutting a canister 200.

The counting device may be attached (e.g. using adhesive) to the top surface 15 of the canister 200, so that again, the overall shape of the canister 200 is not substantially affected by attaching the counting device 104.

Casing of the counting device 104 is shown as transparent for the purposes of this description, so that the component parts of the counting device 104 can be seen. Components which are the same as in counter 101 are given the same reference numbers, e.g. the button switch 3.

The counting device 104 may communicate with the display means, e.g. LCD display 41, via a communications link such as a flexi 50. A flexi usually includes a flexible substrate with copper tracking on the substrate, the copper tracking providing multiple connection paths for e.g. data communication or power transmission respectively. The communications link, e.g. flexi 50, may be electrically connected to components of the counting device 104 at one end, and may include a plug 51 at the other end for removably engaging in a socket 42 of the display module 400.

The plug 51 may be engaged manually in the socket 42 by a user. The plug 51 may be engaged after mounting of the canister 200, to which the counting device 104 is attached, in the housing 301 to form a dispenser according to the present invention.

FIGS. 17a and 17b show the canister, with counting device 104 attached thereto, mounted in the housing 301 to form the dispenser, with the plug 51 engaged with the socket 42. The plug 51 may include a user engagement surface, e.g. a lip 52, to help a user hold the plug 51 during engagement. Furthermore, as can be seen in FIGS. 17a and 18a, the engagement surface, e.g. lip 52, may project from the socket 42, when the plug 51 is engaged with the socket 42, to help the user hold the plug 51 to disengage the plug 51 from the socket 42. Alternatively the plug 51 may automatically engage with the socket 42 upon mounting of the canister 200 in the housing 301. This may be permitted by providing a rigid flexi for the communications link.

FIGS. 18a and 18b are provided without illustration of the housing 301, to clearly show the engagement between the plug 51 and the socket 42.

The counting device 104 may include a battery, e.g. a button battery 2, to provide power to component parts of the counting device 104. The battery 2 may also provide power to components parts of the display module, e.g. the LCD display 41, via e.g. flexi 50.

Alternatively, the display module 400 may include a battery (not shown) to provide power to component parts of the display module, e.g. LCD display 41. The battery of the display module 400 may also provide power to the component parts of the counting device, via e.g. flexi 50.

The counting device 104 may include a memory. The memory may be included in the ASIC 4. The memory may store any information useful to the user of the dispenser, e.g. information about the amount of drug remaining in the canister 200.

It will be apparent to the skilled person from the teaching herein that the invention can be achieved in ways other than those specifically described in the examples, which do not necessarily limit the appended claims.

1. A dispenser for dispensing a dose of a substance, the dispenser having:
a canister for containing the substance, the canister having a delivery portion at one end which includes delivery means through which a dose is deliverable, the delivery portion being mounted in a housing such that relative movement between the canister and the housing actuates the dispenser, and a protruding portion at an end opposite the delivery portion which protrudes from the housing;
a display means for displaying information related to the number of doses delivered by the dispenser; and
an electrical counting device mounted on the protruding portion, the counting device including a switch;
wherein the switch is actuatable by interaction with the housing during relative movement between the canister and the housing.

2. A dispenser according to claim 1, wherein the display means is included in the counting device.

3. A dispenser according to claim 1, wherein the display means is included in a display module attached to the housing.

4. (canceled)

5. A dispenser according to claim 1, wherein the counting device remains outside the housing during actuation.

6. A dispenser according to claim 1, wherein the switch includes a button displaceable by a physical interaction with the housing during relative movement between the canister and the housing.

7. A dispenser according to claim 6, wherein the button is actuatable in the same direction as the relative movement between the counter and the housing.

8. A dispenser according to claim 1, wherein the switch includes a reed switch actuatable by a magnetic interaction with the housing during relative movement between the canister and the housing.

9. A dispenser according to claim 8, wherein the housing has a magnetic strip mounted on it.

10. A dispenser according to claim 1, wherein the housing includes a finger portion extending from its top edge for interacting with the switch.

11. A dispenser according to claim 6, wherein the button is displaceable in a radial direction.

12. (canceled)

13. A dispenser according to claim 8, wherein the finger portion includes a magnetic part.

14. (canceled)

15. A dispenser according to claim 1, wherein the counting device includes a flexible casing for holding the components of the device, the flexible casing being mounted on a flexible substrate for encapsulating the components.

16. A dispenser according to claim 1, wherein the counting device or the display module includes a card battery.

17. A dispenser according to claim 15, wherein the card battery is formed as part of the flexible substrate.

18. A dispenser according to claim 1, wherein the counting device includes a main body for mounting on the end wall of the canister and a leg portion depending from the main body for mounting on the side wall of the protruding portion of the canister.

19.-20. (canceled)

21. A canister for mounting in a housing to form a dispenser, the canister having:
a delivery portion at one end which includes delivery means through which a dose of a substance contained in the canister is deliverable, the delivery portion being mountable in the housing such that relative movement between the canister and the housing actuates the dispenser, the dispenser including a display means for displaying information related to the number of doses delivered by the dispenser; and
a protruding portion at an end opposite the delivery portion for protruding from the housing; wherein the canister has an electrical counting device attached to the protruding portion, the counting device including a switch;
wherein the switch is actuatable by interaction with the housing during relative movement between the canister and the housing.

22. A canister according to claim 1, wherein the display is included in the counting device.

23. A counting device for mounting on a canister to form a canister according to claim 21.

24. A display module for attaching to a housing to form a dispenser according to claim 3.

25.-28. (canceled)

29. A counting device for attaching to a dispenser to record data relating to the number of doses delivered by the dispenser, wherein the counting device includes a reed switch.
